(19) United States
(12) Patent Application Publication
(10) Pub. No.: US 2010/0022629 A1
(43) Pub. Date: Jan. 28, 2010

(54) Dietary Methods and Compositions for Enhancing Metabolism and Reducing Reactive Oxygen Species

(75) Inventors: Cheng Liu, Richmond, CA (US); Yingfei Wei, Berkeley, CA (US)

Correspondence Address: Davis, Agnor, Rapaport & Skalny, LLC, 10211 Wincopin Circle, Suite 600, Columbia, MD, 21044, US

(73) Assignee: Elixirin, Berkeley, CA (US)

(21) Appl. No.: 12/160,888

(22) PCT Filed: Jan. 12, 2007
(86) PCT No.: PCT/US2007/060452

§ 371 Date: Jul. 14, 2008

Related U.S. Application Data
(60) Provisional application No. 60/759,177, filed on Jan. 13, 2006.

(51) Int. Cl. A61K 31/385; A61P 3/00

(52) U.S. Cl. 514/440

(57) ABSTRACT

This invention relates to methods comprising administering to a subject a dietary composition for modifying cellular metabolism, metabolic production of reactive oxygen species and the resulting level of reactive oxygen species. The invention is drawn to a method comprising a combination of carnitine, lipoic acid and polyphenol, which has the effect of enhancing metabolism and reducing oxygen species at the same time. The invention is also drawn to a method of oral administration of carnitine, lipoic acid and polyphenol to a mammalian host, at an effective dose necessary to affect enhanced metabolic processes and reduced oxygen species in animals including humans.

### BACKGROUND

1. Field of the Invention

This invention relates to the use of dietary compositions and methods for modifying cellular metabolism, the metabolic production of reactive oxygen species, and the resulting level of reactive oxygen species.

2. Background of Invention

Numerous lines of evidence suggest that reactive oxygen species are causing cellular damage through oxidation of the cell membrane, cellular proteins, and genomic DNA. There are multiple consequences of the damage caused by reactive oxygen species. Oxidation of the cell membrane reduces the level of non-saturated fatty acids, resulting in lower fluidity of the membrane, oxidation of cellular proteins compromise intracellular enzymatic activities and decrease metabolism, and oxidation of DNA increases the probability of gene mutations. These mechanisms of damage at the cellular level, either each alone or combined, will result in the lowered functions of tissues and organs, and eventually will have an adverse effect on health.

It is also well established that aged cells suffer from reduced level of mitochondrial metabolism (Shigenaga et al., Proc. Natl. Acad. Sci. USA, 91:10771-10778, 1994; Hagen et al., Proc. Natl. Acad. Sci. USA, 95:9562-9566, 1998; Liu et al., Proc. Natl. Acad. Sci. USA, 99(4):2356-2361, 2002; Hagen et al., Ann. NY Acad. Sci. 959:491-507, 2002). The lower level of mitochondrial metabolism means lower production of ATP, the energy source of all cellular activities. Aged cells have decreased activity, resulting in lower function of an organ like heart, lung, or kidney, which are directly related to the health of the mammalian host. Also, Costell et al. have shown an age-dependent decrease in the levels of carnitine present in human muscle samples (Biochem Biophys. Res. Commun. 161(3):1135-1143, 1989).

U.S. Pat. No. 5,916,912 and WO 98/57627 (Ames et al.) disclose a dietary composition for enhancing metabolism and alleviating oxidative stress by oral administration to the host, an effective dosage of a carnitine, such as acetyl-L-carnitine, and an antioxidant, such as lipoic acid. U.S. Patent Application No. 2004/0044046 (Ames) discloses a method of stabilizing R-α-lipoic acid with nicotinamide and using said composition to treat oxidative stress.

L-carnitine is synthesized primarily in the liver and kidneys as a derivative of the amino acid lysine. Under certain conditions, such as high energy demand, the demand for L-carnitine may exceed an individual's capacity to synthesize it, making carnitine a conditionally essential nutrient. Carnitine is approved in the United States as a drug to protect against muscle wasting disease. (Hagen, et al., 2002, Ibid; Linus Pauling Institute Micronutrient Information Center, online information at lpi.oregonstate.edu/infocenter, August 2005). L-carnitine is involved in chaperoning activated acyl-CoA into the mitochondrial matrix for metabolism and chaperoning intermediate compounds out of the mitochondrial matrix to prevent accumulation of the intermediates.

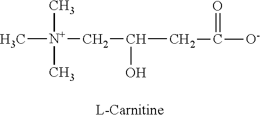

The transport of long-chain fatty acids by L-carnitine into the mitochondrial matrix where they can be metabolized to generate energy requires three enzymes located on the mitochondrial outer and inner membranes. On the outer mitochondrial membrane of skeletal and cardiac muscle cells, carnitine-palmitoyl transferase I (CPTI) catalyzes the formation of acylcarnitine (a fatty acid+L-carnitine) from acyl-CoA (a fatty acid+coenzyme A). A transporter protein called carnitine:acylcarnitine translocase (CT) transports acylcarnitine across the inner mitochondrial membrane. Carnitine-palmitoyl transferase II (CPTII) is associated with the inner mitochondrial membrane and catalyzes the formation of acyl-CoA within the mitochondrial matrix where it can be metabolized through a process called beta-oxidation, ultimately yielding propionyl-CoA and acetyl-CoA.

In regulation of energy metabolism, carnitine acetyl-transferase (CAT) catalyzes the transfer of the acetyl group from acetyl-CoA to L-carnitine, freeing CoA to participate in the conversion of pyruvate to acetyl-CoA, catalyzed by pyruvate dehydrogenase (PDH), a pivitol reaction in glucose metabolism. Decreased levels of free CoA, relative to acetyl-CoA, inhibit the activity of PDH (Linus Pauling Institute Micronutrient Information Center, online information, 2005, Ibid).

Within the mitochondrial matrix, short- and medium-chain fatty acids can be transferred from CoA to L-carnitine, allowing short and medium-chain acyl-carnitines to be exported from the mitochondria. This process provides free CoA needed for energy metabolism, as well as a mechanism to export excess acetyl and acyl groups from the mitochondria. This mechanism may also play a role in the depletion of L-carnitine during the metabolism of certain drugs (Arrigoni-Martelli, E. et al. Drugs Exp. Clin. Res. 27(1):27-49, 2001).

The synthesis of L-carnitine is catalyzed by the concerted action of five different enzymes. This process requires two essential amino acids (lysine and methionine), iron (Fe²⁺), vitamin C, vitamin B6, and niacin in the form of nicotinamide adenine dinucleotide (NAD). One of the earliest symptoms of vitamin C deficiency is fatigue, thought to be related to decreased synthesis of L-carnitine (Linus Pauling Institute Micronutrient Information Center, online information, 2005, Ibid).

The normal rate of L-carnitine biosynthesis in humans ranges from 0.16 to 0.48 mg/kg of body weight/day. Thus, a 70 kg (154 lb) person would synthesize from 11 to 34 mg/day. This rate of synthesis combined with the reabsorption of about 95% of the L-carnitine filtered by the kidneys is enough to prevent deficiency in generally healthy people, including strict vegetarians.

In general L-carnitine appears to be well tolerated. Toxic effects related to L-carnitine overdose have not been reported. L-carnitine supplementation may cause mild gastrointestinal symptoms, including nausea, vomiting, abdominal cramps and diarrhea. Supplements providing more than 3,000 mg/day may cause a “fishy” body odor. Acetyl-L-carnitine has been reported to increase agitation in some Alzheimer's disease patients and to increase seizure frequency and/or severity in some individuals with seizure disorders. Only the L-isomer of carnitine is biologically active. The D-isomer may actually compete with L-carnitine for absorption and transport, increasing the risk of L-carnitine deficiency. Supplements containing a mixture of the D-, and L-isomers (D,L-carnitine) were associated with muscle weakness in patients with kidney disease (Linus Pauling Institute Micronutrient Information Center, online information at lpi.oregonstate.edu/infocenter, August 2005).

Decreases in carnitine levels can be caused by several factors. Myopathic carnitine deficiency: primary myopathic carnitine deficiency is also a genetic disorder in which carnitine deficiency is limited to skeletal and cardiac muscle. Serum L-carnitine levels are generally normal. The symptoms of myopathic carnitine deficiency include muscle pain and progressive muscle weakness and are seen in adults or children (Linus Pauling Institute Micronutrient Information Center, online information, 2005, Ibid).

Secondary carnitine deficiencies may be hereditary or acquired. In all cases, they are characterized by decreased availability of free L-carnitine. In such cases, total L-carnitine levels may be normal, but free L-carnitine levels are decreased.

Hereditary causes: Hereditary causes of secondary carnitine deficiency include genetic defects in amino acid degradation (e.g., propionic aciduria) and lipid metabolism (e.g., medium chain acyl-CoA dehydrogenase deficiency).

Increased L-carnitine loss: hemodialysis, Fanconi syndrome, and the metabolism of some medications may result in substantial L-carnitine loss, resulting in L-carnitine deficiency.

Insufficient L-carnitine synthesis: Malabsorption syndromes and diets that chronically lack L-carnitine and its precursors may increase the risk of secondary carnitine deficiency. Premature infants may be at risk of secondary L-carnitine deficiency when fed soy-based formulas without added L-carnitine. Therefore, it is recommended that non-milk based infant formulas be fortified with the amount of L-carnitine normally found in human milk (11 mg/liter). Although dietary L-carnitine comes mainly from animal sources, even strict vegetarians can generally synthesize enough L-carnitine to prevent deficiencies (Linus Pauling Institute Micronutrient Information Center, online information at lpi.oregonstate.edu/infocenter, August 2005).

The carnitine system can be altered by xenobiotics such as adriamycin (cardiotoxicity), zidovudine (AZT), cisplatin (nephrotoxicity), cephalosporin (nephrotoxicity) and carbapenem (nephrotoxicity) (Arrigoni-Martelli, E. et al. Drugs Exp. Clin. Res. 27(1):27-49, 2001; Linus Pauling Institute Micronutrient Information Center, online information at lpi.oregonstate.edu/infocenter, August 2005; Somani S M et al. Basic Clin. Pharmacol. Toxicol. 86(5):234-241, 2000). Pivalic acid-containing antibiotics used in Europe (pivampicillin, pivmecillinam and pivcephalexin) may also produce secondary L-carnitine deficiencies.

Nucleoside analogues, used in the treatment of HIV infection, including zidovudine (AZT), didanosine (ddI), zalcitabine (ddC) and stavudine (d4T), have been found to have reduced acetyl-carnitine levels in comparison with control groups (Famularo G. et al. AIDS 11:185-190, 1997; Moretti, S et al. Antiox. Redox Sig. 4(3):391-403, 2002). Additionally, patients with HIV-1 infections have been shown to have elevated CD4 and CD8 counts, when administered L-carnitine (Moretti S. et al. Blood 91(10):3817-3824, 1998; Moretti et al., 2002, Ibid). Treatment of patients with HIV with acetyl-L-carnitine has also been shown to be helpful in the treatment of peripheral neuropathies (Scarpini et al., J. Periph. Nerv. Syst. 3(3):227-229, 1998).

The cancer chemotherapy agents, ifosfamide and cisplatin (Somani et al. 2000, Ibid), may increase the risk of secondary L-carnitine deficiency, and there is evidence that L-carnitine supplementation may help prevent cardiomyopathy induced by doxorubicin (adriamycin) therapy (Malarkodi, K P et al., Mol. Cell. Biochem. 247(1-2):9-13, 2003; Arrigoni-Martelli E, et al. 2001, Ibid; Linus Pauling Institute Micronutrient Information Center, online information, 2005, Ibid). Cardiac toxicity is well-known and associated with the anthracyline drugs, such as doxorubicin and daunorubicin, with mitoxantrone, epirubicin, idarubicin and esorubicin also being cardiotoxic. These toxicities in include myocardial ischemia, pericarditis, arrhythmias ECG changes, cardiomyopathies, and angina. The acute affects, such as ECG changes and arrythmias, can occur within hours of a bolus administration. Subacute (weeks to months) and chronic (up to five years) are clinically significant as they are related to myocardial cell damage (Fischer et al. The Cancer Chemotherapy Handbook 4^{th} Ed., Mosby-Year Book, Inc. St. Louis, Mo., pp. 484-485, 1989). Carnitine has also been found to reduce serum levels of proinflammatory cytokines IL-6 and tumor necrosis factor (TNFf), along with a reduction of reactive oxygen species and glutathione peroxidase levels in patients with advanced cancer (Mantovani G et al. Free Rad. Res. 37(2):213-223, 2003).

Carnitine use in humans has led to an improvement in symptoms of patients with heart disease; reports have demonstrated improvements in chest pain, loss of breath, fatigue and so forth. In humans, the administration of L-carnitine immediately after the diagnosis of MI has resulted in improved clinical outcomes in several clinical trials. In one trial, half of 160 men and women diagnosed with a recent MI were randomly assigned to receive 4 grams/day for 12 months of L-carnitine in addition to standard pharmacological treatment. After one year of treatment, there was an improvement is heart rate, improved exercise capacity, systolic and diastolic arterial pressure, improvement in lipid pattern, angina attacks were less frequent and mortality was significantly lower in the L-carnitine supplemented group (1.2% vs. 12.5%) (Davini P. et al., Drugs Exp. Clin. Res. 18(8):355-365, 1992; Witte K K A, et al. J. Am Coll. Cardiol. 37:1765-1774, 2001; Singh R B et al., Postgrad. Med. J. 72:45-50, 1996; Colonna P. and Iliceto S. Am. Heart J. 139:2(Pt3):S124-130, 2000; Hagen et al. 2002, Ibid).

Improvements in nephrotoxcity and kidney disease have been seen in humans using carnitine. A lack of carnitine in hemodialysis patients is caused by insufficient carnitine synthesis and especially by its loss during dialysis. L-Carnitine and many of its precursors are removed from the circulation during dialysis. Impaired L-carnitine synthesis by the kidneys may also contribute to the potential for carnitine deficiency in patients with end-stage renal failure on hemodialysis. The U.S. Food and Drug Administration (FDA) has approved the use of L-carnitine in hemodialysis patients not only for the treatment, but also for the prevention of carnitine depletion in dialysis patients. Furthermore, clinical guidelines developed by both American and European nephrological societies suggest a trial with carnitine supplementation that may establish efficacy in selected dialysis patients who do not adequately respond to standard therapy for certain conditions, such as severe and persistent muscle cramps or hypotension during dialysis, lack of energy, myopathy, cardiomyopathy, and anemia of uremia unresponsive to or requiring large doses of erythropoietin. (Guarnieri G. et al. Am. J. Kideny Dis. 38(4 Suppl 1):S63-67, 2001). Carnitine depletion may lead to a number of conditions observed in dialysis patients, including muscle weakness and fatigue, plasma lipid abnormalities, and refractory anemia. A review that examined the results of 18 randomized trials, including a total of 482 dialysis patients, found that L-carnitine treatment was associated with improved hemoglobin levels in studies performed before recombinant erythropoietin (EPO) was routinely used to treat anemia in dialysis patients, and that L-carnitine treatment decreased EPO dose and resistance to EPO in studies performed when patients routinely received EPO (Hurot J M et al., Soc. Nephrol. 13(3):708-714, 2002). Some studies demonstrated regular carnitine supplementation of hemodialysis patients can improve their lipid metabolism, protein nutrition, red blood cell count, and antioxidant status. (Vesela E. et al., Nephron. 88(3):218-223, 2001). In general, intravenous L-carnitine therapy (1-2 grams) at the end of a dialysis session has been recommended for patients on hemodialysis. Oral administration of L-carnitine (1-3 grams/day in divided doses) is more practical for patients on peritoneal dialysis (Ahmad S. Semin. Dial. 14(3):209-217, 2001). Additionally, in patients with end stage renal disease, therapy with either carnitine or propionylcarnitine has been shown to increase the exercise capacity of patients (Brass and Haitt, J. Amer. Coll. Nutr. 17(3):207-215, 1998). Acetyl-L-carnitine has also been shown to be useful in improving pain, nerve fiber cluster regeneration and vibration perception in patients with diabetic neuropathy (Sima et al., Diab. Care 28(1):89-94, 2005).

### Lipoic Acid

Lipoic acid is a coenzyme in mitochondria that is involved in carbohydrate utilization necessary for the production of ATP and maintaining oxidative balance such as intracellular glutathione levels.

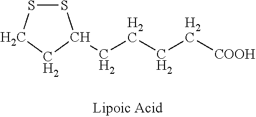

Alpha-lipoic acid is also known as thioctic acid, 1,2-dithiolane-3-pentanoic acid, 1,2-dithiolane-3-valeric acid and 6,8-thioctic acid. Alpha lipoic acid can be present in two enantiomeric forms (R- and S-). The (R-) form of lipoic acid can present stability problems when stored, which can be solved by complexing R-α-lipoic acid with nicotinamide (U.S. Patent application 2004/0044046). Lipoic acid can be present in a reduced form, dihydrolipoate (DHLA) and is an excellent antioxidant capable of interacting with most forms of reactive oxygen species, recycling other antioxidants and additionally reducing oxidized disulfide groups in biological systems.

Alpha-lipoic acid has also been found to be an inhibitor of NF-kappa B activation in human T cells and endothelial cells, which can have wide-ranging effects on the immune system. Suzuki et al. (Biochim Biophys. Res. Commun. 189(3):1709-1715, 1992) found that incubation of Jurkat T cells with the antioxidant, alpha-lipoic acid prior to the stimulation of cells was found to inhibit NF-kappa B activation induced by tumor necrosis factor-alpha or by phorbol ester. The inhibitory action of alpha-lipoic acid was found to be very potent. Their results indicated that alpha-lipoic acid may be effective in AIDS therapeutics, as antioxidants which eliminate reactive oxygen species should block the activation of NF-kappa B and subsequently HIV transcription. Suzuki et al. suggested that antioxidants can be used as therapeutic agents for AIDS. Zhang and Frei (FASEB J. 15(13):2423-2432, 2001) reported that the metabolic thiol antioxidant alpha-lipoic acid may be of therapeutic value in pathologies associated with redox imbalances, such as atherogenesis thought to be caused by endothelial activation and monocyte adhesion influenced in part by oxidative stress. Preincubation of human aortic cells with lipoic acid dependently inhibited TNF-alpha-induced adhesion of human monocytic THP-1 cells, as well as mRNA and protein expression of E-selectin, vascular cell adhesion molecule 1 and intercellular adhesion molecule 1. Lipoic acid also strongly inhibited TNF-alpha-induced mRNA expression of monocyte chemoattractant protein-1 but did not affect expression of TNF-alpha receptor 1. Furthermore, LA dose-dependently inhibited TNF-alpha-induced IkappaB kinase activation, subsequent degradation of IkappaB, the cytoplasmic NF-kappaB inhibitor, and nuclear translocation of NF-kappaB. The data demonstrated that clinically relevant concentrations of LA, inhibit adhesion molecule expression in aortic endothelial cells and monocyte adhesion by inhibiting the IkappaB/NF-kappaB signaling pathway at the level, or upstream, of IkappaB kinase.

Alpha lipoic acid has also been found to have therapeutic potential to counteract cumulative oxaliplatin related peripheral neuropathy in patients with advanced colorectal cancer (Gedlicka et al., J Clin. Oncol. 20(15):3359-3361, 2002). Neuropathies are well-known in the use of platinum containing anti-cancer drugs, such as cisplatin and carboplatin as well as with vinka alkaloids, cytarabine, and taxol, among others. Nephrotoxicity is also seen clinically in cancer patients treated with various chemotherapeutic agents (Fischer et al. The Caner Chemotherapy Handbook, Ibid, pp. 475-495, 1993). Age-dependent protection by lipoic acid against nephrotoxicity in cisplatin-treated rats was reported by Somani et al. (Bas. Clin. Pharmacol. Tox., 86(5):234-241, 2000). Malarkodi et al. found an increase in peroxidated lipids upon administration with adriamycin, a common therapeutic agent used in treating various forms of cancer. The study reported nephroprotection when rats were treated with lipoic acid prior to the use of adriamycin (Molec. Cell. Biochem. 247(1-2):9-13, 2003).

Lipoic acid has also been shown to provide beneficial results in the improvement of microcirculation in hypertensive and diabetic patients, including the improvement of peripheral diabetic neuropathy (Midaoui et al., Hypertension, 39:303-307, 2002; Midaoui et al., Am. J. Hypertens. 16(3):173-179, 2003; Haak et al., Exp. Clin. Endocrinol. Diabetes 108(3):168-174, 2000; Heitzer et al., Free Radic. Biol. Med. 31(1):53-61, 2001; Androne et al. In Vivo, 14(2):327-330, 2000; Zeigler et al., Diabetologia, 38(12):1425-1433, 1995; Reljanovic et al., Free Rad. Res. 31(3):171-179, 1999; Jacob et al., Exp. Clin. Endocrinol. Diabetes, 104(3):284-288, 1996; Jacob et al., Free Radic Biol. Med. 27(3-4):309-314, 1999). In addition, Alpha lipoic acid has been reported to decrease oxidative stress in diabetic patients with poor glycemic control (Borcea et al., Free Radic. Biol. Med. 26(11-12):1495-500, 1999) and improves cellular glucose utilization in patients with Type II diabetes (Konrad et al., Diabetes Care 22:280-287, 1999). Additionally, lipoic acid has been found to be protective against atherosclerosis in a quail model (Shih J C, Fed. Proc. 42(8):2494-2497, 1983).

Moreover, lipoic acid has been found to prevent cataract formation in newborn rats (Maitra et al., Free Rad. Biol. Med., 18:823-829, 1995).

Lipoic acid is also involved in metal chelation and detoxification as reported by Sigel et al. (Arch. Biochem. Biophys. 187:208-214, 1978); Devasagayam et al., (Chem-Biol Interations 86:79-92, 1993); Berkson B M, (Med. Klin. 94 Suppl. 3:84-89, 1999); Nagy et al., (Clin. Invest. 72:794-798, 1994); and Sabeel et al., (Mycopathologica, 131:107-114, 1995).

There exists a need in the art for a method of optimizing health benefits for subjects using a dietary composition that can achieve a better health benefit than any of the previously-reported methods. In addition, currently available supplements with a carnitine and lipoic acid combination (see U.S. Pat. No. 5,916,912 and WO 98/57627, both Ames, et al.), does not provide enough anti-oxidation activity, which may result in suboptimal health benefits for the treated subject.

### SUMMARY OF INVENTION

The present invention is drawn to a method of optimizing health benefits in a subject comprising using a composition comprising a combination of carnitine, lipoic acid, and polyphenol which has the effect of enhancing metabolism and reducing reactive oxygen species at the same time.

In one embodiment of the invention, the combination of carnitine, lipoic acid and polypenol will increase the metabolic rate of damaged cells of a mammalian host due to mycocardial infarction, heart disease, atherosclerosis, chemotherapy, stroke, kidney disease, diabetes, neurodegerative diseases such as Parkinson's disease or Alzheimer's disease, or poor nutrition, without increasing the production of reactive oxygen species. Therefore, when the combination is fed to older animals, the animals will have improved metabolism at the cellular level and a resulting reduction of oxidative stress. The animals will consequently experience reversal of several gross indicators of aging, including cognitive activity, and capacity of physical movement.

The present invention includes a method of oral administration of carnitine, lipoic acid and polypenol to a mammalian host at an effective dose necessary to affect enhanced metabolic processes and reduced reactive oxygen species in animals, including humans.

### BRIEF DESCRIPTION OF THE FIGURES

FIG. 1 of 2 depicts spatial memory in rats subjected to a water maze test, after treatment with carnitine, lipoic acid, and/or polyphenol.

FIG. 2 of 2 depicts the measurement of oxidation levels of DNA in rats following a treatment with carnitine, lipoic acid, and/or polyphenol.

### DETAILED SUMMARY OF THE INVENTION

The present invention is drawn to a method of optimizing health benefits in a subject comprising using a composition comprising a combination of carnitine, lipoic acid, and polyphenol. The method may be used in a subject which includes animals such as farm animals, pets, and research animals, including, but not limited to bovine, ovine, porcine, equine, or avian animals; in feline or canine, or other animals such as ferrets, guinea pigs, rats, mice, llamas, alpacas, emus, water buffalo, bison, fish, reptiles, zoological specimens, and so forth. The composition may also be used in humans as a dietary supplement or clinically, as needed by a patient.

The present invention can also be used in the culture of microorganisms or animal cells as an additive for maximizing culture conditions in a laboratory or industrial setting (see Jay et al., U.S. Pat. No. 5,536,645, “Nutritive medium for the culture of microorganisms”).

The present invention is drawn to a method of optimizing health benefits in a subject comprising the use of a composition comprising carnitine and its derivatives, intermediates and/or precursors, which are normal mitochondrial metabolites that facilitate transport of fatty acids to the mitochondria. Examples of carnitine and its derivatives, which are encompassed by the present invention include: acetyl-L-carnitine, mercapto acyl-carnitines, actetyl carnitine esters, mercapto carnitine esters, niconinoyl carnitine and derivatives, alkoxy-acyl derivatives of carnitine, alkoxy-acyl derivatives of carnitine, N-alkylamides of d(+)-carnitine, and compositions thereof.

Increasing carnitine levels in a host is expected to enhance mitochondrial activity, therefore leading to higher levels of metabolism. The effective dosage of carnitine of the invention is at least about 0.1 mg/kg host per day, at least about 1 mg/kg host per day, at least about 10 mg/kg host per day, at least about 50 mg/kg host per day, at least about 100 mg/kg host per day, at least about 200 mg/kg host per day, or at least about 250 mg/kg host per day. Dosages of the carnitine can also be administered in the range of at least about 0.1 mg/kg to at least about 1 g/kg, in the range of at least about 1 mg/kg to at least about 500 mg/kg more or in the range of at least about 4 mg/kg to at least about 50 mg/kg of body weight per day, although variations will necessarily occur depending on the formulation, host, and so forth. It is understood that the dosages of carnitine may be greater or lesser than the dosages described herein, as an artisan in the field would appreciate and determine to be effective, and be within the scope of the present invention.

The present invention is also drawn to a method comprising the use of a composition comprising lipoic acid and its derivatives, intermediates and/or precursors. The effective dosage of lipoic acid of the invention is at least about 0.01 mg/kg host per day, at least about 0.1 mg/kg host per day, at least about 1 mg/kg host per day, at least about 10 mg/kg host per day, at least about 50 mg/kg host per day, at least about 100 mg/kg host per day, at least about 200 mg/kg host per day, or at least about 250 mg/kg host per day. Dosages of the lipoic acid can also be administered in the range of at least about 0.01 mg/kg to at least about 0.1 mg/kg, in the range of at least about 0.1 mg/kg to at least about 1 mg/kg, in the range of at least about 0.5 mg/kg to at least about 100 mg/kg more or in the range of at least about 1 mg/kg to at least about 10 mg/kg of body weight per day, although variations will necessarily occur depending on the formulation, host, and so forth. It is understood that the dosages of lipoic acid may be greater or lesser than the dosages described herein, as an artisan in the field would appreciate and determine to be effective, and be within the scope of the present invention.

The present invention is also drawn to a method comprising the use of a composition comprising polyphenol, derivatives of polyphenol and precursors thereof. Polyphenol is a family of strong anti-oxidants that have been found in a variety of natural products such as green tea (Katiyar et al., J. Leuk. Biol. 69:719-726, 2001). Polyphenols such as epigallocathechin-3 galate (EGCG), epigallocathechin, epicathichin-3, (−)-epigallocatechin-3-gallate, theaflavin, and thearubigin derivatives, intermediates and precursors and the like are included in the invention. Polyphenols are substances found in various teas and wines. Polyphenols are known as cathechins, including epigallocathechin-3 galate (EGCG), epigallocathechin and epicathichin-3. Catechins are the main polyphenolic flavonols of green tea. Theaflavins and thearubigins are constituents in black teas. It has been shown that EGCG increases tyrosine phosphorylation of the insulin receptor and insulin receptor substrate-1 (IRS-1). It has been reported that black and green tea polyphenols has been found to have antidiabetic effects (Waltner-Law et al., J. Biol. Chem. 277(38):34933-34940, 2002), effects in inflammatory responses (Katiyar and Mukhtar, Carcinogenesis, 18(10):1911-1916, 1997), has beneficial effects in lipid metabolism (Yee et al., Mol. Cell. Biochem. 229(1-2):85-92, 1001; Yang and Koo, Pharmacol. Res. 35(6):505-512, 1997), in attenuating hypertension (Negishi et al., J. Nutr. 134:38-42, 2004), in the treatment of obesity (Chantre and Lairon, Phytomedicine, 9(1):3-8, 2002), and in cancer treatments and prevention (Suganuma et al., Mutat. Res. 428(1-2):339-344, 1999; Katiyar and Mukhtar J. Cell Biochem Suppl. 27:59-67, 1997; Katiyar et al., Carcinogenesis, 18(3):497-502, 1997; Mukhtar and Ahmed, Toxicol. Sci. 52(Suppl):111-117, 1999; Sadzuka et al., Clin. Cancer Res. 4(1):153-156, 1998; Suganuma et al., Cancer Res. 59:44-47, 1999). Inhibition of intestinal tumorigenesis in Apc^{min/+} mice by (−)-epigallocatachin-3-gallate was reported by Ju et al. (Canc. Res. 65(22):10623-10631, Nov. 15, 2005). The Apc^{min/+} intestinal tumorigenesis model is recognized to be a model for human intestinal cancer. The APC gene is a tumor suppressor gene that functions as a negatrive regulator of β-catenin by ubiquitin-dependent proteasomes. Truncated or mutated Apc proteins, encoded by the Apc gene is found in human colon cancers and usually are unable to bind to β-catenin.

The effective dosage of polyphenol of the invention is at least about 0.01 mg/kg host per day, at least about 0.03 mg/kg host per day, at least about 1 mg/kg host per day, at least about 10 mg/kg host per day, at least about 100 mg/kg host per day, at least about 200 mg/kg host per day, or at least about 250 mg/kg host per day. Dosages of the polyphenol can also be administered in the range of at least about 0.01 mg/kg to at least about 1 g/kg, in the range of at least about 0.05 mg/kg to at least about 500 mg/kg more or in the range of at least about 0.03 mg/kg to at least about 10 mg/kg of body weight per day, although variations will necessarily occur depending on the formulation, host, and so forth. It is understood that the dosages of polyphenol may be greater or lesser than the dosages described herein, as an artisan in the field would appreciate and determine to be effective, and be within the scope of the present invention.

A method comprising the use of a composition of a combination of these three components will have the cellular effect of enhancing ATP production while reducing reactive oxygen species. Measurements regarding parameters of aging in a host are well known in the art and include, but are not limited to, activities and behavior such as social interactions, motivation, grooming, mental acuity and memory, sexual activity, physical strength, energy level, immune responses, cardiovascular symptoms, as well as physical appearances such as hair or coat condition, wound repair, cellular and molecular lesions, muscle strength and tone, kidney function, and the like.

A method comprising the compositions of the present invention comprises administering a dosage of the compositions of the invention in an amount that is therapeutically effective or in an amount determined to optimize health benefits in a subject in need thereof.

The effective dosage may be in a combination of carnitine in the range of at least about 0.1 mg/kg to at least about 1 g/kg, in the range of at least about 1 mg/kg to at least about 500 mg/kg more or in the range of at least about 4 mg/kg to at least about 50 mg/kg of body weight per day; lipoic acid can also be administered in the range of at least about 0.01 mg/kg to at least about 0.1 mg/kg, in the range of at least about 0.1 mg/kg to at least about 1 mg/kg, in the range of at least about 0.5 mg/kg to at least about 100 mg/kg more or in the range of at least about 1 mg/kg to at least about 10 mg/kg of body weight per day; and polyphenol can also be administered in the range of at least about 0.01 mg/kg to at least about 1 g/kg, in the range of at least about 0.05 mg/kg to at least about 500 mg/kg more or in the range of at least about 0.03 mg/kg to at least about 10 mg/kg of body weight per day, although variations will necessarily occur depending on the formulation, host, and so forth. The effective dosage can be at least about 5 mg/kg host/day of carnitine, at least about 5 mg/kg host/day of lipoic acid, and at least about 5 mg/kg host/day of polyphenol for use in animals.

The dosage administered depends upon the age, health and weight of the subject, type of previous or concurrent treatment, if any, frequency of treatment, and the nature of the effect desired. The compositions of the invention can be administered by any means that achieve their intended purposes. Amounts and regimens for the administration of the composition according to the present invention can be determined readily by those with ordinary skill in the art. Administration of the composition of the present invention can also optionally be included with previous, concurrent, subsequent or adjunctive therapy in a clinical setting or as part of a dietary regimen.

In addition to the active compounds, a composition of the present invention can also contain suitable carriers acceptable for dietary use and/or pharmaceutical use comprising excipients and auxiliaries which facilitate processing of the active compounds into preparations which can be used pharmaceutically or as a dietary supplement. Suitable formulations for oral administration include hard or soft gelatin capsules, dragees, pills, tablets, including coated tablets, elixirs, suspensions, syrups or inhalations and controlled release forms thereof. Preferably, the preparations, particularly those preparations which can be administered orally and which can be used for the preferred type of administration, such as tablets; dragees, and capsules; softgels; blisters; functional foods, such as power bars, gums, candies, and the like; and functional drinks, such as soft drinks, juices, milks, soy drinks, power drinks, and the like. Drinks such as tea, herbal preparations, coffees and the like are also included in the invention.

Suitable excipients are, for example, fillers such as saccharide, lactose or sucrose, dextrose, sucralose (SPLENDA®), aspartame, saccharine, mannitol or sorbitol; cellulose preparations and/or calcium phosphates, such as tricalcium phosphate or calcium hydrogen phosphate; as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone, and may also include preparations comprising natural honey or derivatives. If desired, disintegrating agents can be added such as the above-mentioned starches and also carboxymethyl starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions can be used, which can optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl cellulose phthalate are used. Dyestuffs or pigments can be added to the tablets or dragee coatings, for example, for identification or in order to characterize combinations of active compound doses.

Other preparations which can be used orally include push-fit capsules made of gelatin, as well as soft, sealed capsules made of gelatin and a plasticizer such as glycerol or sorbitol. The push-fit capsules can contain the active compounds in the form of granules which can be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils or liquid paraffin. In addition, stabilizers can be added.

Solid dosage forms in addition to those formulated for oral administration include rectal suppositories. The composition of the present invention can also be administered in the form of an implant when compounded with a biodegradable slow-release carrier. Suitable injectable solutions include intravenous subcutaneous and intramuscular injectable solutions. Alternatively, the composition of the invention may be administered in the form of an infusion solution or as a nasal inhalation or spray. Alternatively, the composition of the present invention can be formulated as a transdermal or transmucosal patch for continuous release of the active ingredient.

Possible preparations that can be used rectally include, for example, suppositories that consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

A formulation for systemic administration according to the invention can be formulated for enteral, parenteral or topical administration. Indeed, all three types of formulation can be used simultaneously to achieve systemic administration of the active ingredient.

Suitable formulations for parenteral administration include aqueous solutions of the active compounds in water-soluble form, for example, water-soluble salts. In addition, suspensions of the active compounds as appropriate oily injection suspensions can be administered. Suitable liphophilic solvents or vehicles include fatty oils, such as sesame oil, or synthetic fatty acid esters, such as ethyl oleate or triglycerides. Aqueous injection suspensions that can contain substances that increase the viscosity of the suspension include, for example, sodium carboxymethyl cellulose, sorbitol, and/or dextran. Optionally, the suspension can also contain stabilizers.

Suitable formulations for topical administration include creams, gels, jellies, mucilages, pastes and ointments.

The invention provides administratively convenient formulations of the compositions including dosage units incorporated into a variety of containers. Convenient unit dosage containers include metered sprays, measured liquid containers, measured powdered containers and the like. The compositions can be combined and used in combination with other therapeutic or prophylactic agents. For example, the compounds may be advantageously used in conjunction with other antioxidants, free radical scavengers, and mixtures thereof, or other mixtures as known in the art, (e.g. Goodman & Gilman, The Pharmacological Basis of Therapeutics, 9^{th} Ed., 1996, McGraw-Hill). In another embodiment, the invention provides the subject compounds in the form of one or more pro-drugs, which can be metabolically converted to the subject compounds by the recipient host. A wide variety of pro-drug formulations are known in the art.

Compositions of the present invention are manufactured in a manner which is itself known, for example, by means of conventional mixing, granulating, dragee-making, dissolving, or lyophilizing processes. Thus, preparations for oral use can be obtained by combining the active compounds with solid excipients, optionally grinding the resulting mixture, and processing the mixture of granules, after adding suitable auxiliaries, if desired or necessary, to obtain tablets or dragee cores.

The methods of the invention includes administration of the compositions of the invention in can also further comprise anti-viral agents such as, but not limited to, AZT (RETROVIR®, zidovudine), 3TC (EPIVIR®, lamivudine), ddI (VIDEX®, didanosine), ddC (HIVID®, zalcitabine, 2′-3′-dideoxycytidine), D4T (ZERIT®, stavudine), abacavir, nevirapine, delavirdine, efavirenz, saquinavir, ritonavir, 3′-azido-2′,3′-dideoxyadenosine (AZA), indinavir, nelfinavir, amprenavir, adefovir, TAMIFLU®, FLU-MIST® and hydroxyurea. GP-41 is an HIV transmembrane protein which has been shown to be essential for the virus to fuse with and infect healthy cells. Anti-viral peptides are included in the invention and refers to peptides that inhibit viral infection of cells, by, for example, inhibiting cell-cell fusion or free virus infection. The route of infection may involve membrane fusion, as occurs in the case of enveloped viruses, or some other fusion event involving viral and cellular structures. Peptides that inhibit viral infection by a particular virus may be referenced with respect to that particular virus, e.g., anti-HIV peptide, anti-RSV peptide, etc. Antifusogenic peptides are peptides demonstrating an ability to inhibit or reduce the level of membrane fusion events between two or more entities, e.g., virus-cell or cell-cell, relative to the level of membrane fusion that occurs in the absence of the peptide. In particular, anti-viral and antifusogenic peptides include the DP107 and DP178 peptides and analogs thereof, as well as peptides comprised of amino acid sequences from other (non-HIV) viruses that correspond to the gp41 region of HIV from which DP107 and DP178 are derived, and that exhibit anti-viral or anti-fusogenic activity. These peptides can exhibit anti-viral activity against not only HIV, but other viruses including human respiratory syncytial virus (RSV), human parainfluenza virus (HPV), measles virus (MeV) and simian immunodeficiency virus (SIV). In particular, anti-HIV peptides refer to peptides that exhibit anti-viral activity against HIV, including inhibiting CD4+ cell infection by free virus and/or inhibiting HIV-induced syncytia formation between infected and uninfected CD4+ cells. Anti-SIV peptides are peptides that exhibit anti-viral activity against SIV, including inhibiting of infection of cells by the SIV virus and inhibiting syncytia formation between infected and uninfected cells. Anti-RSV peptides are peptides that exhibit anti-viral activity against RSV, including inhibiting mucous membrane cell infection by free RSV virus and syncytia formation between infection and uninfected cells. Anti-HPV peptides are peptides that exhibit anti-viral activity against HPV, including inhibiting infection by free HPV virus and syncytia formation between infected and uninfected cells. Anti-MeV peptides are peptides that exhibit anti-viral activity against MeV, including inhibiting infection by free MeV virus and syncytia formation between infected and uninfected cells.

Compositions included in the methods of the invention can also include additional therapeutic agents such as, but not limited to hydrophilic drugs, hydrophobic drugs, hydrophilic macromolecules, cytokines, peptidomimetics, peptides, proteins, toxoids, sera, antibodies, vaccines, nucleosides, nucleotides, nucleoside analogs, genetic materials and/or combinations thereof.

Additional suitable antiviral agents for optimal use with the compositions of the present invention can include, but are not limited to, AL-721 (lipid mixture) manufactured by Ethigen Corporation and Matrix Research Laboratories; Amphotericin B methyl ester; Ampligen (mismatched RNA); anti-AIDS antibody; 1 AS-101 (heavy metal based immunostimulant); Betaseron (β-interferon) manufactured by Triton Biosciences; butylated hydroxytoluene; Carrosyn (polymannoacetate); Castanospermine; Contracan (stearic acid derivative); Creme Pharmatex (containing benzalkonium chloride); CS-87 (5-unsubstituted derivative of Zidovudine), Cytovene (ganciclovir); dextran sulfate; D-penicillamine (3-mercapto-D-valine; Foscarnet (trisodium phosphonoformate); fusidic acid manufactured by Leo Lovens; glycyrrhizin (a constituent of licorice root); HPA-23 (ammonium-21-tungsto-9-antimonate) manufactured by Rhone-Poulenc Sante; human immune virus antiviral developed by Porton Products International; Ornidyl (eflornithine); nonoxinol; pentamidine isethionate (PENTAM-300); Peptide T (octapeptide sequence) manufactured by Peninsula Laboratories; phenytoin; Ribavirin; Rifabutin (ansamycin); CD4-IgG2 or other CD4-containing or CD4-based molecules; T-20; Trimetrexate; SK-818 (germanium-derived antiviral); suramin and analogues thereof; UA001 manufactured by Ueno Fine Chemicals Industry; and Wellferon, α-interferon).

Methods of the present invention includes the use of the compositions of the present invention which can also further comprise immunomodulators. Suitable immunomodulators for optional use the compositions of the present invention can include, but are not limited to: ABPP (Bropririmine); Ampligen (mismatched RNA); anti-human interferon-α-antibody; anti-AIDS antibody; AS-101 (heavy metal based immunostimulant; ascorbic acid and derivatives thereof; interferon-β; Carrosyn (polymannoacetate); Ciamexon; cyclosporin; cimetidine; CL-246,738; colony stimulating factors, including GM-CSF; dinitrochlorobenzene; HE2000; interferon-α; inteferon-γ; glucan; hyperimmune gamma-globulin (Bayer); IMREG-1 (leukocyte dialyzate) and IMREG-2; immuthiol (sodium diethylthiocarbamate); interleukin-1; interleukin-2 (IL-2); isoprinosine (inosine pranobex); Krestin (Sankyo Corp.); LC-9018; lentinan (Ajinomoto/Yamanouchi Corporations); LF-1695 (Fournier Corporation); methionine-enkephalin; Minophagen C; muramyl tripeptide, MTP-PE (Ciba-Geigy); naltrexone (Trexan); Neutropin, RNA immunomodulator (Nippon Shingaku); Remune (Immune Response Corporation); Reticulose; shosaikoto and ginseng; thymic humoral factor; TP-05 (Thymopentin); Thymosin factor 5 and Thymosin 1; Thymostimulin; TNF (tumor necrosis factor); and vitamin B preparations.

Examples of therapeutic agents that can be used in the compositions of the present invention include, but are not limited to, other antineoplastic agents, analgesics and anti-inflammatory agents, anti-anginal agents, antihelmintics, anti arrythmic agents, anti-arthritic agents, anti-asthma agents, anti-bacterial agents, anti-viral agents, antibiotics, anti-coagulants, anti-depressants, anti-diabetic agents, anti-epileptic agents, anti-emetics, anti-fungal agents, anti-gout agents, anti-hypertensive agents, anti-malarial agents, antimigraine agents, anti-muscarinic agents, anti-parkinson's agents, anti-protozoal agents, anti-thyroid agents, thyroid therapeutic agents, anti-tussives, anxiolytic agents, hypnotic agents, neuroleptic agents, β-blockers, cardiac inotropic agents, corticosteroids, diuretics, gastrointestinal agents, histamine H-receptors antagonists, immunosuppressants, keratolytics, lipid regulating agents, muscle relaxants, nutritional agents, cytokines, peptidomimetics, peptides, proteins, toxoids, sera, sedatives, sex hormones, sex hormone antagonists or agonists, stimulants antibodies, vaccines, nucleosides, nucleoside analogs and genetic materials. Amphiphilic therapeutic agents and nutritional agents can also be included.

Antineoplastic agents which can be used in the method of the invention include, but are not limited to anticancer agents known in the art include busulphan, chlorambucil, hydroxyurea, ifosfamide, mitomycin, mitotane, chlorambucil, mechlorethamine, carmustine, lomustine, carmustine, herceptin, cyclophosphamide, nitrosoureas, fotemustine, vindescine, etoposide, daunorubicin, adriamycin, paclitaxel, docetaxel, streptozocin, dactinomycin, doxorubicin, idarubicin, plicamycin, pentostatin, mitotoxantrone, valrubicin, cytarabine, fludarabine, floxuridine, clardribine, methotrexate, mercaptopurine, thioguanine, capecitabine, irinotecan, dacarbazine, asparaginase, gemcitabine, altretamine, topotecan, procarbazine, vinorelbine, pegaspargase, vinblastin, rituxan, vinblastine, tretinoin, teniposide, fluorouracil, fluorodeoxyuridine, melphalan, bleomycin, platinum containing agents such as cisplatin, carboplatin, salicylates, aspirin, piroxicam, ibuprofen, indomethacin, naprosyn, diclofenac, tolmetin, ketoprofen, nambuetone, oxaprozin, nonselective cycclooxygenase inhibitors such as nonsteroidal anti-inflammatory agents (NSAIDS), and selective cyclooxygenase-2 (COX-2) inhibitors. Other chemotherapeutic agents useful in the application of this invention include: decarbazine, lonidamine, piroxantrone, anthrapyrazoles, camptothecin, 9-aminocamptothecin, 9-nitrocamptothecin, camptothecin-11 (irinotecan), topotecan, bleomycin, the vinca alkaloids and their analogs, such as vincristine, vinorelbine, vindesine, vintripol, vinxaltine, ancitabine; 6-aminochrysene, topoisomerase inhibitors, such as VP16 and camptothecins; and navelbine. Other compounds useful in the invention include: 1,3-bis(2-chloroethyl)-1-nitrosurea (carmustine or BCNU), epirubicin, aclarubicin, bisantrene(bis(2-imidazolen-2-ylhydrazone)-9,10-anthracenedicarboxaldehyde, edatrexate, muramyl tripeptide, muramyl dipeptide, lipopolysaccharides, vidarabine and its 2-fluoro derivative, resveratrol, retinoic acid and retinol, carotenoids, taxol, taxotere and tamoxifen.

Also included in the methods of the invention are compositions also comprising antihypertensive agents that can be used to treat hypertension, including but not limited to enalapril, acebutolol, and doxazosin. Acebutolol is in a class of drugs called beta-blockers, which affect the heart and circulatory system. Doxazosin is a member of the alpha blocker family of drugs used to lower blood pressure in subjects with hypertension. Doxazosin is also used to treat symptoms of benign prostatic hyperplasia (BPH). Diuretics, such as furosemide (LASIX®), chlorothiazide (DIURIL®), hydrochlorothiazide (ESIDRIX®, HYDRODIURIL®), and spironolactone (ALDACTONE®). The method also includes the use of beta blockers that are used to reduce high blood pressure. Propranolol (INDERAL®), metoprolol (LOPRESSOR®), atenolol (TENORMIN®), bisoprolol (ZEBETA®), and carvedilol (COREG®). Also included are ACE inhibitors, which include benazepril (LOTENSIN®), captopril (CAPOTEN®), enalapril (VASOTEC®), fosinopril (MONOPRIL®), lisinopril (PRINIVIL®, ZESTRIL®), moexipril (UNIVASC®), perindopril (ACEON®), quinapril (ACCUPRIL®), ramipril (ALTACE®), and trandolapril (MAVIK®). Also included in the invention, but not limited to compositions comprising angiotensin II receptor antagonists, including valsartan (DIOVAN®), candesartan (ATACAND®), eprosartan (TEVETEN®), irbesartan (AVAPRO®), losartan potassium (COZAAR®), olmesartan (BENICAR®), and telmisartan (MICARDIS®). Most of these drugs are also commercially available combined with a diuretic. The method of the invention also includes use of calcium channel blockers including amlodipine (NORVASC®), bepridil (VASCOR®), diltiazem (CARDIZEM®, DILACOR XR®, TIAZAC®), felodipine (PLENDIL®), isradipine (DYNACIRC®), nicardipine (CARDENE®), nimodipine (NIMOTOP®), nisoldipine (SULAR®), and verapamil (CALAN®, COVERA-HS®, ISOPTIN®, VERELAN®). Some calcium channel blockers are available combined with an ACE inhibitor in a single dosage, including LEXXEL®, LOTREL®, and TARKA®.

Also included is methylprednisolone, a synthetic steroid that suppresses acute and chronic inflammation. In addition, methylprednisolone stimulates gluconeogenesis, increases catabolism of proteins and mobilization of free fatty acids, potentiates vascular smooth muscle relaxation by beta adrenergic agonists, and may alter airway hyperactivity. Methylprednisolone is also a potent inhibitor of the inflammatory response.

The invention can include “blood-brain barrier” compositions, including proteins which can traverse this barrier through protein transduction. Small sections of these proteins (10-16 residues long), i.e. BBB peptides, are responsible for this transduction. RGD peptides for conjugation to tissues or fixed endogenous proteins in accordance with the present invention includes a sequence of amino acids, preferably naturally occurring L-amino acids and glycine, having the following formula: R₁-Arg-Gly-Asp-R₂. In this formula, R₁ and R₂ represent an amino acid or a sequence of more than one amino acid or a derivatized or chemically modified amino acid or more than one derivatized or chemically modified amino acids.

Insulinotropic peptides (ITPs) are peptides with insulinotropic activity. Insulinotropic peptides stimulate, or cause the stimulation of, the synthesis or expression of the hormone insulin. Such peptides include precursors, analogues, fragments of peptides such as Glucagon-like peptide, exendin 3 and exendin 4 and other peptides with insulinotropic activity. Glucagon-Like Peptide (GLP) and GLP derivatives are intestinal hormones which generally simulate insulin secretion during hyperglycemia, suppresses glucagon secretion, stimulates (pro) insulin biosynthesis and decelerates gastric emptying and acid secretion. Some GLPs and GLP derivatives promote glucose uptake by cells but do not simulate insulin expression as disclosed in U.S. Pat. No. 5,574,008 which is hereby incorporated by reference.

### EXAMPLES

### Example 1

### Morris Water Spatial Memory in Rats

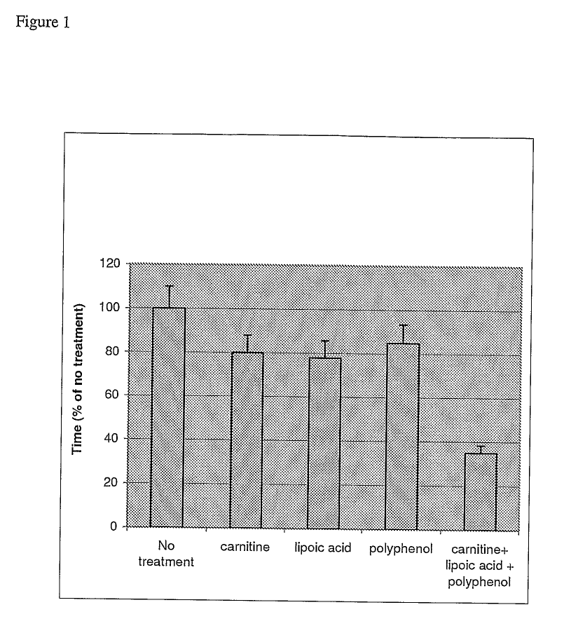

FIG. 1 of 2 depicts measurements of spatial memory in (Fischer 344 male rats) subjected to a water maze test, after treatment with carnitine, lipoic acid, and/or polyphenol. The rats that were treated with a combination of carnitine, lipoic acid, and polyphenol demonstrated greater than 50% faster mastery of the water maze than the rats treated with any of the components alone (approximately 38% faster for the combination over no treatment versus approximately 80%, 79% and 82% for carnitine, lipoic acid and polyphenol alone, respectively). The Morris maze task tests spatial memory by requiring rats to find a submerged platform in a pool of water using external visual cues (Morris, R. 1984; J. Neurosci. Methods, 11:47-60; Schenk, F. and Morris, R, Exp. Brain Res. 58:11-28, 1985). The rats in the experimental group (5 each) were fed either 0.5% (wt/vol) acetyl-L-carnitine in water, 1.0% (wt/vol) polyphenol in water; 0.2% (wt/wt) lipoic acid in AIN93M diet, or a combination of the above. Rats were acclimated for one week before experiments. Rats were fed for the above treatment for 7 weeks before tests. Trials (4 consecutive days, 4 trials per day) were conducted and the results averaged. The standard deviation is around 20%. Control rats were fed with water and AIN93M diet only.

### Oxidation Levels of DNA

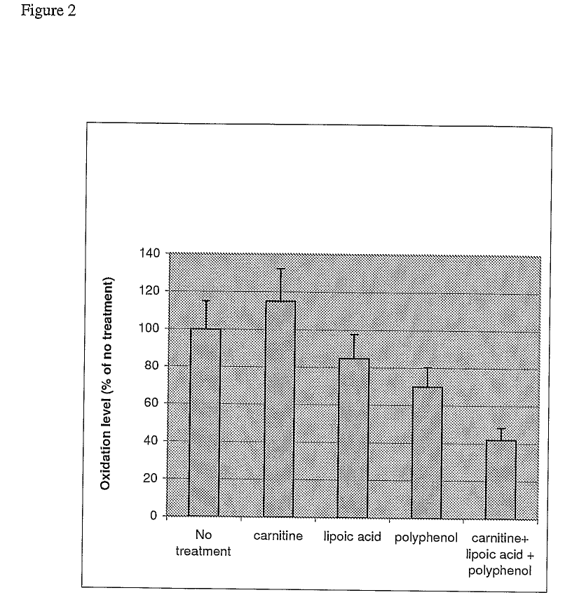

FIG. 2 of 2 demonstrates that the combination treatment of carnitine, lipoic acid, and/or polyphenol resulted in a lowering of oxidative stress in rats, as measured by the oxidation level of DNA. The combination treatment of carnitine, lipoic acid, and polyphenol resulted in an oxidation level of 41% compared with rats having no treatment. Rats treated with carnitine, lipoic acid, and polyphenol alone resulted in DNA oxidation levels of approximately 112%, 82%, and 65%, respectively. The rats in the experimental group (5 each) were fed either 0.5% (wt/vol) acetyl-L-carnitine in water, 1.0% (wt/vol) polyphenol in water; 0.2% (wt/wt) lipoic acid in AIN93M diet, or a combination of the above. Rats were acclimated for one week before experiments. Rats were fed for the above treatment for 7 weeks before tests. Control rats were fed with water and AIN93M diet only. For assaying DNA oxidation, rats from each treatment group (5 animals per group) was anesthetized with ether and perfused with paraformaldehyde. The brain was removed and postfixed for paraffin sections. Sections of hippocampus were incubated with anti-8-hydroxy-2′-deoxyguanosine/8-hydroxyguanosine and visualized by using standard immunocytochemical methods. Quantification of oxidation was done as described in Liu, J K, et al., (Proc. Natl. Acad. Sci. USA, 99:1356-2361, 2002). Values are a mean of 5 animals. The standard deviation is about 15%.

### Clinical Improvement of Human Patients

In a clinical setting, six (6) chemotherapy patients receiving a combination of carnitine, lipoic acid and polyphenol demonstrated a reduction in nausea and fatigue and an increase in appetite over the course of their treatment over a one-month period of time. The dosages of the chemotherapy agents used in human patients depended on the clinical situation of the patients and the side effects caused by the chemotherapeutic agents used in their anti-cancer therapy. The patients took from 2 tablets/day to 6 tablets/day. Each tablet contained 200 mg acetyl-carnitine, 83 mg alpha lipoic acid, and 6.6 mg EGCG. Improvements in side effects were noted within one to two days of treatment and showed an improvement in all parameters measured.

Heart patients receiving a combination of carnitine, lipoic acid and polyphenol demonstrated an improvement in chest pain (angina), loss of breath and fatigue. The dosages of the therapeutic agents used in human patients depended on the clinical situation of the patients and the side effects caused by the therapeutic agents used in their cardiac therapy. The patients took from 2 tablets/day to 6 tablets/day. Each tablet contained 200 mg acetyl-carnitine, 83 mg alpha lipoic acid, and 6.6 mg EGCG.

Having now fully described this invention, it will be understood to those of ordinary skill in the art that the same can be performed within a wide and equivalent range of conditions, formulations, and other parameters without affecting the scope of the invention or any embodiment thereof. All patents and publications cited herein are incorporated by reference in their entirety.

We claim:

1. A method of improving health benefits of a subject comprising administering to a subject a composition comprising carnitine, lipoic acid and polyphenol.

2. The method of claim 1, wherein said composition comprises carnitine in the range of at least about 4 mg/kg to at least about 50 mg/kg subject per day, and lipoic acid in the range of at least about 0.1 mg/kg to at least about 10 mg/kg subject per day, and polyphenol in the range of at least about 0.03 mg/kg to at least about 1 mg/kg subject per day.

3. The method of claim 2, wherein the carnitine is in the form of actyl-L-carnitine.

4. The method of claim 2, wherein the lipoic acid is in the form of alpha-lipoic acid.

5. The method of claim 2, wherein the polyphenol is in the form of (−)-epigallocatechin-3-gallate.

6. The method of claim 1, wherein said composition comprises carnitine in the range of at least about 0.005 mg/l to at least about 100 mg/l, and lipoic acid in the range of 0.005 mg/l to at least about 100 mg/l, and polyphenol in the range of at least about 0.005 mg/l to at least about 100 mg/l.

7. The method of claim 1, wherein said composition is in the form selected from the group consisting of hard gelatin capsule, soft gelatin capsule, dragee, pill, tablet, coated tablet, powder, elixir, suspension, syrup, inhaler, drink, candy, gum, and power bar.

8. The method of claim 2, wherein said subject is selected from the group consisting of bovine, ovine, porcine, equine, avian, feline, canine, ferrets, guinea pigs, rats, mice, llamas, alpacas, emus, water buffalo, bison, fish, reptiles, and zoological specimens.

9. A method of modifying cellular metabolism comprising administering to a subject a composition comprising carnitine, lipoic acid and polyphenol, wherein said subject is a cancer patient also receiving chemotherapeutic agents.

10. The method of claim 9, wherein said composition comprises carnitine in the range of at least about 4 mg/kg to at least about 50 mg/kg subject per day, and lipoic acid in the range of at least about 0.1 mg/kg to at least about 10 mg/kg subject per day, and polyphenol in the range of at least about 0.03 mg/kg to at least about 1 mg/kg subject per day.

11. The method of claim 10, wherein the carnitine is in the form of actyl-L-carnitine.

12. The method of claim 10, wherein the lipoic acid is in the form of alpha-lipoic acid.

13. The method of claim 10, wherein the polyphenol is in the form of (−)-epigallocatechin-3-gallate.

14. The method of claim 10, wherein said subject is selected from the group consisting of bovine, ovine, porcine, equine, avian, feline, canine, ferrets, guinea pigs, rats, mice, llamas, alpacas, emus, water buffalo, bison, fish, reptiles, and zoological specimens.

15. A method of modifying cellular metabolism comprising administering to a subject a composition comprising carnitine, lipoic acid and polyphenol, wherein said subject is a heart disease patient.

16. The method of claim 15, wherein said medium comprises carnitine in the range of 0.005 mg/l to at least about 100 mg/l, and lipoic acid in the range of at least about 0.005 mg/l to at least about 100 mg/l, and polyphenol in the range of at least about 0.005 to at least about 100 mg/l.

17. The method of claim 16, wherein the carnitine is in the form of actyl-L-carnitine.

18. The method of claim 16, wherein the lipoic acid is in the form of alpha-lipoic acid.

19. The method of claim 16, wherein the polyphenol is in the form of (−)-epigallocatechin-3-gallate.
